(19) United States
(12) Patent Application Publication
(10) Pub. No.: US 2015/0322350 A1
(43) Pub. Date: Nov. 12, 2015

(54) Fischer-Tropsch Based Gas to Liquids Systems and Methods

(71) Applicant: Siluria Technologies, Inc., San Francisco, CA (US)

(72) Inventors: Rahul Iyer, Kensington, CA (US); Bipinkumar Patel, Richmond, TX (US); Erik Scher, San Francisco, CA (US)

(21) Appl. No.: 14/707,466

(22) Filed: May 8, 2015

Related U.S. Application Data
(60) Provisional application No. 61/991,361, filed on May 9, 2014.

(51) Int. Cl. C10G 2/00; C01B 3/34; C07C 5/327

(52) U.S. Cl. CPC C10G 2/32; C10G 2/34; C07C 5/327; C01B 3/34; C01B 2203/0216; C01B 2203/062; C01B 2203/1241

(57) ABSTRACT

A method for generating hydrocarbon compounds containing at least two carbon atoms (C₂₊ compounds) comprises directing a natural gas feed stream from a non-Fischer Tropsch process and comprising methane and C₂₊ compounds to at least one separation unit to separate the methane from the C₂₊ compounds. The separated C₂₊ compounds are directed to a fractionation unit to separate the separated C₂₊ compounds into individual streams. The separated methane is directed to a synthesis gas (syngas) unit to partially oxidize the methane to hydrogen (H₂) and carbon monoxide (CO), which are subsequently directed to a Fischer-Tropsch unit comprising a Fischer-Tropsch catalyst. In the Fischer-Tropsch unit, the hydrogen and carbon monoxide react to generate C₂₊ compounds in a Fischer-Tropsch process. The C₂₊ compounds are directed to the fractionation unit to separate the generated C₂₊ compounds into streams each comprising a subset of the generated C₂₊ compounds.

### CROSS-REFERENCE

This application claims the benefit of U.S. Provisional Application No. 61/991,361, filed May 9, 2014, which application is incorporated herein by reference.

### BACKGROUND

Gas to liquids (GTL) is a process to convert natural gas or other gaseous hydrocarbons into longer-chain hydrocarbons, such as gasoline or diesel fuel. In a typical GTL process, methane-rich gases are converted into liquid synthetic fuels either via direct conversion—using non-catalytic processes that convert methane to methanol in one step—or via synthesis gas (syngas) as an intermediate, such as in the Fischer-Tropsch process. Combinations are also possible such as methane to methanol followed by methanol to olefins (methanol to gasoline, MTG).

### SUMMARY

Recognized herein is the need for efficient and commercially viable gas to liquids (GTL) systems and methods for converting methane into to higher chain hydrocarbons, such as hydrocarbon compounds with two or more carbon atoms (also “C₂₊ compounds” herein), such as olefins and/or alkanes.

The present disclosure provides systems and methods for generating C₂₊ compounds from methane in a multi-step process that comprises (i) converting methane to synthesis gas (“syngas”) and (ii) converting the syngas to C₂₊ compounds in a Fischer-Tropsch process. A Fischer-Tropsch (FT) process can start with partial oxidation (PO), steam methane reforming, or autothermal reforming (ATR) of methane to hydrogen gas and carbon monoxide, and in some cases carbon dioxide and water. The ratio of carbon monoxide to hydrogen can be adjusted using a water gas shift reaction or other m of separation and/or purification. Syngas can be reacted with the aid of a Fischer-Tropsch catalyst to yield liquid hydrocarbons, including C₂₊ compounds. Examples of Fischer-Tropsch catalysts include but are not limited to iron-based and cobalt-based heterogeneous catalysts. Catalysts can include those prepared with high temperature synthesis or with low temperature synthesis.

Methane can typically be difficult to store. Processes that generate methane as a byproduct routinely burn the methane. However, the present disclosure provides approaches for generating useful products from methane by retrofitting processes with the requisite unit operations to convert the methane to higher molecular weight hydrocarbons. In some cases, this is possible by making use of existing unit operations (e.g., separations units).

An aspect of the present disclosure provides a method for generating hydrocarbon compounds containing at least two carbon atoms (C₂₊ compounds), comprising: (a) directing a natural gas feed stream comprising methane and C₂₊ compounds to at least one separation unit to separate said methane from said C₂₊ compounds, wherein said natural gas feed stream is from a non-Fischer-Tropsch process; (b) directing at least a portion of said methane separated in (a) to a syngas unit, and in said syngas unit partially oxidizing said methane to hydrogen (H₂) and carbon monoxide (CO); (c) directing said hydrogen and carbon monoxide to a Fischer-Tropsch unit comprising a Fischer-Tropsch catalyst, and in said Fischer-Tropsch unit reacting said hydrogen and carbon monoxide in a Fischer-Tropsch process to generate a product stream comprising C₂₊ compounds; and (d) directing said C₂₊ compounds separated in (a) and said product stream comprising said C₂₊ compounds generated in (c) to a fractionation unit, and in said fractionation unit separating said C₂₊ compounds into individual streams.

In some embodiments of aspects provided herein, compounds comprising at least 10 carbon atoms are removed from said product stream prior to directing said product stream to said fractionation unit in (e). In some embodiments of aspects provided herein, the compounds comprising at least 10 carbon atoms are removed using a refrigerant shared with said fractionation unit. In some embodiments of aspects provided herein, the fractionation unit comprises one or more distillation columns. In some embodiments of aspects provided herein, the fractionation unit comprises at least one cryogenic separation system. In some embodiments of aspects provided herein, the at least one cryogenic separation system is a propylene-based cryogenic separation system. In some embodiments of aspects provided herein, the fractionation unit comprises at least one pressure swing adsorption unit. In some embodiments of aspects provided herein, the natural gas feed stream has a C₂₊ compound concentration that is less than 50%. In some embodiments of aspects provided herein, the natural gas feed stream has a C₂₊ compound concentration that is less than 40%. In some embodiments of aspects provided herein, the natural gas feed stream has a C₂₊ compound concentration that is less than 30%. In some embodiments of aspects provided herein, the natural gas feed stream has a C₂₊ compound concentration that is less than 20%. In some embodiments of aspects provided herein, the natural gas feed stream has a C₂₊ compound concentration that is less than 10%. In some embodiments of aspects provided herein, the natural gas feed stream has a C₂₊ compound concentration that is less than 5%. In some embodiments of aspects provided herein, the method further comprises combining at least a subset of the individual streams from (d) with the individual streams from (e). In some embodiments of aspects provided herein, the method further comprises directing C₂₊ compounds generated in (c) to a light hydrocarbon recovery unit, wherein the light hydrocarbon recovery unit generates tail gas. In some embodiments of aspects provided herein, the method further comprises directing the tail gas to the at least one separation unit. In some embodiments of aspects provided herein, the method further comprises directing hydrogen and/or carbon monoxide recovered in the fractionation unit to the Fischer-Tropsch unit. In some embodiments of aspects provided herein, the method further comprises, prior to (d), directing the C₂₊ compounds generated in (c) to at least one separation unit to separate the C₂₊ compounds from non-C₂₊ impurities. In some embodiments of aspects provided herein, the non-C₂₊ impurities comprise one or more of methane, H₂, and CO. In some embodiments of aspects provided herein, the method further comprises directing the non-C₂₊ impurities to the syngas unit or the Fischer-Tropsch unit. In some embodiments of aspects provided herein, the method further comprises directing the C₂₊ compounds from the at least one separation unit to the fractionation unit. In some embodiments of aspects provided herein, the method further comprises, prior to (b), directing a portion of the methane separated from the C₂₊ compounds for use as pipeline gas. In some embodiments of aspects provided herein, the Fischer-Tropsch catalyst comprises at least one transition metal. In some embodiments of aspects provided herein, the Fischer-Tropsch catalyst comprises one or more elemental metals selected from the group consisting of cobalt, iron, ruthenium, and nickel. In some embodiments of aspects provided herein, the method further comprises directing the methane separated in (b) for use as pipeline gas. In some embodiments of aspects provided herein, the natural gas feed stream further comprises hydrogen and carbon monoxide. In some embodiments of aspects provided herein, the fractionation unit separates the C₂₊ compounds into hydrocarbon compounds comprising from 2 to 30 carbon atoms. In some embodiments of aspects provided herein, the fractionation unit separates the C₂₊ compounds into hydrocarbon compounds comprising from 2 to 15 carbon atoms. In some embodiments of aspects provided herein, the fractionation unit separates the C₂₊ compounds into hydrocarbon compounds comprising from 2 to 10 carbon atoms. In some embodiments of aspects provided herein, the method further comprises, prior to (a), retrofitting the non-Fischer Tropsch process with a Fischer Tropsch process comprising the syngas unit and the Fischer Tropsch unit. In some embodiments of aspects provided herein, the non-Fischer-Tropsch process comprises the fractionation unit. In some embodiments of aspects provided herein, the method further comprises, prior to (e), separating hydrocarbon compounds containing at least 30 carbon atoms from the generated C₂₊ compounds. In some embodiments of aspects provided herein, the hydrocarbon compounds containing at least 30 carbon atoms are selected from the group consisting of diesel and wax. In some embodiments of aspects provided herein, the method further comprises, prior to (e), directing the product stream to a methanation unit to convert CO, CO₂, and H₂ in the product stream to methane. In some embodiments of aspects provided herein, the method further comprises, prior to (e), directing the product stream to a cracking unit to convert alkanes among the C₂₊ compounds to alkenes. In some embodiments of aspects provided herein, the Fischer-Tropsch unit and the fractionation unit are located within about 5 miles of each other. In some embodiments of aspects provided herein, the Fischer-Tropsch unit and the fractionation unit are located within about 1 mile of each other. In some embodiments of aspects provided herein, the Fischer-Tropsch unit produces less than about 500 kilotons per annum (kTa) of the product stream. In some embodiments of aspects provided herein, the Fischer-Tropsch unit produces less than about 100 kilotons per annum (kTa) of the product stream. In some embodiments of aspects provided herein, the Fischer-Tropsch unit produces less than about 50 kilotons per annum (kTa) of the product stream.

An aspect of the present disclosure provides a system for generating hydrocarbon compounds containing at least two carbon atoms (C₂₊ compounds), comprising: (a) at least one separation unit that (i) accepts a natural gas feed stream comprising methane and C₂₊ compounds from a non-Fischer-Tropsch process and (ii) separates said methane from said C₂₊ compounds; (b) a syngas unit downstream of and fluidically coupled to said at least one separation unit, wherein said syngas unit (i) accepts at least a portion of said methane separated in said at least one separation unit and (ii) partially oxidizes said methane to hydrogen (H₂) and carbon monoxide (CO); (c) a Fischer-Tropsch unit downstream of and fluidically coupled to said syngas unit, wherein said Fischer-Tropsch unit comprises a Fischer-Tropsch catalyst for facilitating a Fischer-Tropsch process, and wherein said Fischer-Tropsch unit (i) accepts said H₂ and said CO and (ii) reacts said H₂ and CO in said Fischer-Tropsch process to generate a product stream comprising C₂₊ compounds; and (d) a fractionation unit downstream of and fluidically coupled to said at least one separation unit and said Fischer-Tropsch unit, wherein said fractionation unit (i) accepts said C₂₊ compounds separated in said at least one separation unit and said product stream comprising said C₂₊ compounds generated in said Fischer-Tropsch unit and (ii) separates said C₂₊ compounds into individual streams.

In some embodiments of aspects provided here, said fractionation unit comprises at least one cryogenic separation system. In some embodiments of aspects provided herein, said at least one cryogenic separation system comprises a propylene-based cryogenic separation system. In some embodiments of aspects provided herein, said fractionation unit comprises at least one pressure swing adsorption unit. In some embodiments of aspects provided herein, said natural gas feed stream has a C₂₊ compound concentration that is less than 50%. In some embodiments of aspects provided herein, said natural gas feed stream has a C₂₊ compound concentration that is less than 30%. In some embodiments of aspects provided herein, said fractionation unit separates said C₂₊ compounds into hydrocarbon compounds comprising from 2 to 30 carbon atoms. In some embodiments of aspects provided herein, said non-Fischer-Tropsch processes comprises said fractionation unit. In some embodiments of aspects provided herein, said system further comprises a methanation unit that converts CO, carbon dioxide (CO₂), and H₂ in said product stream to methane. In some embodiments of aspects provided herein, said system further comprises a cracking unit that converts alkanes among said C₂₊ compounds to alkenes. In some embodiments of aspects provided herein, said Fischer-Tropsch unit and said fractionation unit are located within about 5 miles of each other. In some embodiments of aspects provided herein, said Fischer-Tropsch unit produces less than about 500 kilotons per annum (kTa) of said product stream. In some embodiments of aspects provided herein, said Fischer-Tropsch unit produces less than about 100 kilotons per annum (kTa) of said product stream. In some embodiments of aspects provided herein, said Fischer-Tropsch unit produces less than about 50 kilotons per annum (kTa) of said product stream.

Another aspect of the present disclosure provides a computer readable medium comprising machine executable code that, upon execution by one or more computer processors, implements any of the methods above or elsewhere herein.

Another aspect of the present disclosure provides a computer system comprising one or more computer processors and memory coupled thereto. The memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” and “FIGS.” herein), of which:

FIG. 1 shows a Fischer-Tropsch (FT) process;

FIG. 2 shows a natural gas processing system that is configured and adapted to generate hydrocarbons using an FT process;

FIGS. 3A and 3B show methanation systems that can be used with systems of the present disclosure;

FIG. 4 shows a separation system that may be employed for use with systems and methods of the present disclosure;

FIG. 5 shows another separation system that may be employed for use with systems and methods of the present disclosure;

FIG. 6 shows another separation system that may be employed for use with systems and methods of the present disclosure;

FIG. 7 shows another separation system that may be employed for use with systems and methods of the present disclosure;

FIG. 8 is a process flow diagram of a system that comprises a hydrogenation unit and a demethanizer unit, which can be employed for small scale and world scale olefin production;

FIG. 9 shows a computer system that is programmed or otherwise configured to regulate FT processes and systems of the present disclosure;

FIG. 10 shows an example of integrating an MTG process with a gas processing plant; and

FIG. 11 shows an example of a system for converting natural gas to gasoline involving the retrofit of a natural gas processing plant with a methanol to gasoline process.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “Fischer-Tropsch process,” as used herein, generally refers to a process that involves or substantially involves the generation of hydrocarbons from hydrogen (H₂) and carbon monoxide (CO). A Fischer-Tropsch (FT) process can be facilitated by a heterogeneous catalyst.

The term “non-Fischer Tropsch process,” as used herein, generally refers to a process that does not involve or substantially involve the generation of hydrocarbons from H₂ and CO. Examples of processes that may be non-Fischer Tropsch processes include hydrocarbon separation in oil refineries, natural gas liquids separations processes, steam cracking of ethane and steam cracking of naphtha.

The terms “C₂₊” and “C₂₊ compound,” as used herein, generally refer to a compound comprising two or more carbon atoms, e.g., two carbon atoms (C₂), three carbon atoms (C₃), etc. C₂₊ compounds include, without limitation, alkanes, alkenes, alkynes and aromatics containing two or more carbon atoms. In some cases, C₂₊ compounds include aldehydes, ketones, esters and carboxylic acids. Examples of C₂₊ compounds include ethane, ethylene, acetylene, propane, propene, butane, butene, etc.

The term “non-C₂₊ impurities,” as used herein, generally refers to material that does not include C₂₊ compounds. Examples of non-C₂₊ impurities, which may be found in certain FT product streams, include nitrogen (N₂), oxygen (O₂), water (H₂O), argon (Ar), hydrogen (H₂) carbon monoxide (CO), carbon dioxide (CO₂) and methane (CH₄).

The term “syngas,” as used herein, generally refers to synthesis gas, which is a mixture of CO and H₂.

The term “unit,” as used herein, generally refers to a unit operation, which is a basic step in a process. Unit operations involve a physical change or chemical transformation, such as separation, crystallization, evaporation, filtration, polymerization, isomerization, transformation, and other reactions. A given process may require one or a plurality of unit operations to obtain the desired product from the starting materials, or feedstocks.

The term “small scale,” as used herein, generally refers to a system that generates less than or equal to about 250 kilotons per annum (KTA) of a given product, such as an olefin (e.g., ethylene).

The term “world scale,” as used herein, generally refers to a system that generates greater than about 250 KTA of a given product, such as an olefin (e.g., ethylene). In some examples, a world scale olefin system generates at least about 1000, 1100, 1200, 1300, 1400, 1500, or 1600 KTA of an olefin.

### Fischer-Tropsch Processes and Systems

An aspect of the present disclosure provides methods for forming C₂₊ compounds using Fischer-Tropsch processes. Such methods can employ the integration of a Fischer-Tropsch process in a non-Fischer Tropsch system or process, which can include retrofitting the non-Fischer Tropsch system or process with equipment to enable the formation of C₂₊ compounds using inputs from the non-Fischer Tropsch system or process.

In a Fischer Tropsch (FT) process, one or more hydrocarbons are generated upon the reaction of hydrogen (H₂) and carbon monoxide (CO). The reaction can be facilitated by a heterogeneous catalyst, such as iron or cobalt with other elements. Hydrocarbons that can be generated by an FT process include C₂₊ compounds.

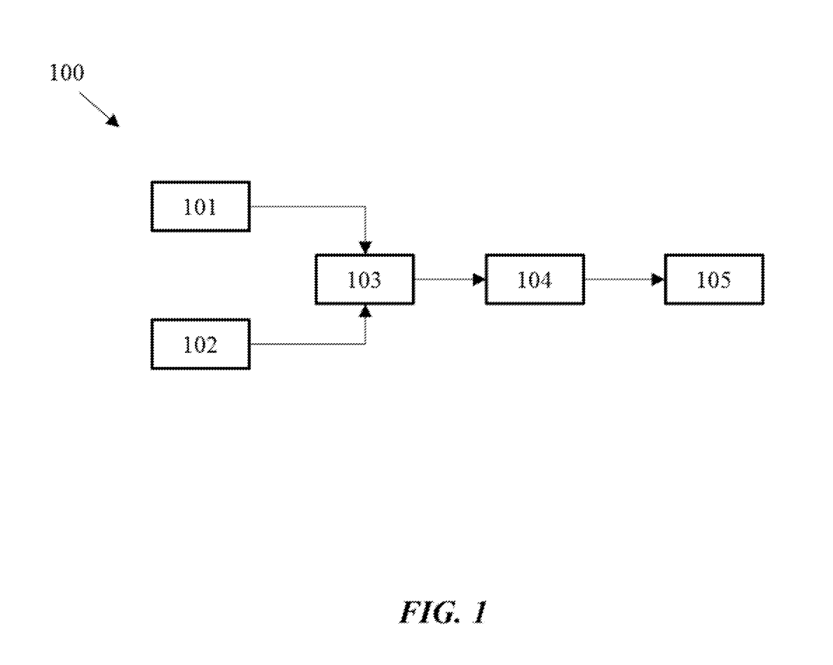

FIG. 1 shows an FT process 100, as may be employed for use with methods (or processes) and systems of the present disclosure. FT processes can include but are not limited to steam methane reforming, autothermal reforming (ATR), and partial oxidation (PO). The FT process 100 includes a source of methane (CH₄) 101, source of water (H₂O) 102, a gas synthesis unit 103, at least one Fischer-Tropsch (FT) reactor 104, and a separation system 105. The process can also include a source of oxygen (e.g., air, oxygen-enriched air, or oxygen). Inputs and outputs into respective units are indicated by arrows. The source of methane 101 can be a natural gas source, such as a natural gas feed stream comprising CH₄ and in some cases C₂₊ compounds and non-C₂₊ impurities. The source of methane can include one or more separation units to separate CH₄ from any C₂₊ compounds and non-C₂₊ impurities.

During use, methane from the source of methane 101 and water from the source of water 102 are directed into the gas synthesis unit 103, which reacts CH₄ and H₂O to generate CO and H₂, for example through the following reaction: CH₄+H₂O→CO+3H₂. Oxygen from a source of oxygen can also be directed into the gas synthesis unit. Next, CO and H₂ from the gas synthesis unit 103 are directed to the FT reactor 104, where CO and H₂ react in an FT process to form hydrocarbons, including C₂₊ compounds. The hydrocarbons can be directed to the separation system 105, which separates the hydrocarbons into streams each comprising a substrate of the C₂₊ compounds and in some cases non-C₂₊ impurities.

The separation system 105 can include at least 1, 2, 3, 4, 5, 6, 7, 8, 9, 10, 20, 30, 40, or 50 separation units, which can be in series and/or parallel. Each separation unit can be configured to effect the separation of an input stream into separate streams each comprising a subset of the components in the input stream. Examples of separation units include distillation units, absorption units, vapor-liquid separation units, and cryogenic separation units. In some examples, the separation system 105 includes at least 1, 2, 3, 4, 5, 6, 7, 8, 9, 10, 20, 30, 40, or 50 distillations units.

The source of methane 101 can include C₂₊ compounds. In some cases, the source of methane 101 has a C₂₊ compound concentration that is less than about 50%, 40%, 30%, 20%, 10%, 5%, or 1%.

The at least one FT reactor 104 can include at least 1, 2, 3, 4, 5, 6, 7, 8, 9, 10, 20, 30, 40, or 50 FT reactors. In some cases, the at least one FT reactor 104 includes at least 2, 3, 4, 5, 6, 7, 8, 9, 10, 20, 30, 40, or 50 FT reactors in series. As an alternative, the at least one FT reactor 104 includes at least 2, 3, 4, 5, 6, 7, 8, 9, 10, 20, 30, 40, or 50 FT reactors in parallel. As another alternative, the at least one FT reactor 104 includes at least 2, 3, 4, 5, 6, 7, 8, 9, 10, 20, 30, 40, or 50 FT reactors, at least some of which are in series and some of which are in parallel. If multiple FT reactors are employed in series, each FT reactor can include the same or a different catalyst as another FT reactor. For example, one FT reactor can include a catalyst to effect formation of hydrocarbons having between two and ten carbon atoms, and another FT reactor can include a catalyst to effect the formation of hydrocarbons having greater than ten carbon atoms.

An FT reactor can include a heterogeneous catalyst. The catalyst may be in the form of a honeycomb, packed (or fixed) bed, fluidized bed, microchannel reactor, tubular reactor, bubble-column reactor, or other bed or reactor types. FT catalysts that can be employed for use with systems and methods of the present disclosure can comprise at least one metal or metallic material, such as a transition metal selected from iron (Fe), ruthenium (Ru), nickel (Ni), and cobalt (Co), which may be present in the form of an oxide, carbide, elemental metal, alloy, or a combination thereof. In some examples, the catalyst may comprise from about 10% to about 60% cobalt (based on the weight of the metal as a percentage of the total weight of the catalyst precursor), or from about 35% to about 50% of cobalt, or from about 40% to about 44% of cobalt, or about 42% of cobalt. In some cases, the cobalt is present as CoO and/or Co₃O₄. Examples of catalysts that may be employed for use with FT reactors of the present disclosure are provided in U.S. Patent Publication Nos. 2009/0010823 and 2014/0045954, and U.S. Pat. Nos. 7,084,180, 7,722,833 and 8,188,153, which are entirely incorporated herein by reference.

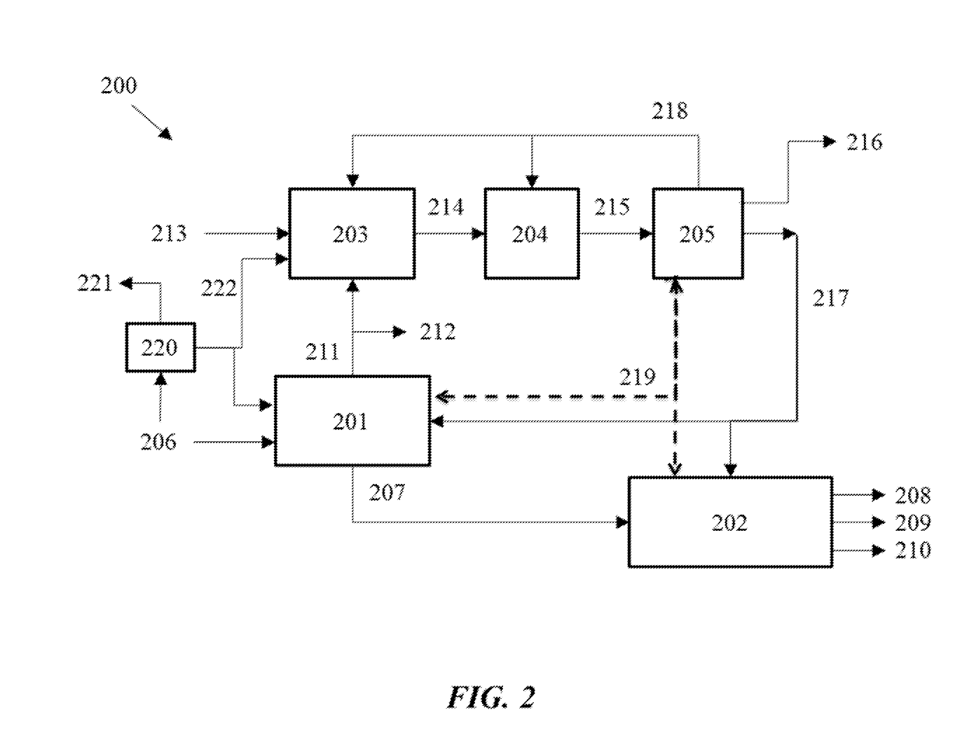

FIG. 2 shows a system 200 that is configured and adapted to generate hydrocarbons using an FT process. The FT process can be integrated into (e.g., retrofitted with) a non-FT process, such as a natural gas processing plant as shown here. The system 200 includes a first separation module 201 and a second separation module 202 downstream of the first separation module 201. The first separation module 201 can be a fractionation module. The process 200 further includes a gas synthesis module 203 downstream of the first separation module 202, an FT module 204 downstream of the gas synthesis module 203, and a third separation module 205 downstream of the FT module 204. Each of the separation modules 201, 202 and 205 can include at least 1, 2, 3, 4, 5, 6, 7, 8, 9, 10, 20, 30, 40, or 50 separation units, such as described above in the context of FIG. 1. In some examples, the first separation module can include one or more distillation units, cryogenic separation units, and/or recycle split vapor (RSV) units.

During use, feed stream 206 comprising methane is directed to the first separation module 201, which separates methane from other components of the feed stream 206, such as C₂₊ compounds. Separated C₂₊ compounds 207 can be directed to the second separation module 202, which separates the C₂₊ compounds into individual streams, such as, for example, a first product stream 208, second product stream 209, and third product stream 210. The product streams 208-210 can include different (average) distributions of hydrocarbons. For example, the first product stream 208 can include hydrocarbons having between two and five carbons atoms, the second product stream 209 can include hydrocarbons having between five and ten carbons atoms, and the third product stream 210 can include hydrocarbons having greater than ten carbons atoms.

Methane from the first separation module 201 can be directed to the gas synthesis module 203 along stream 211. A portion of the methane in stream 211 can be removed along stream 212 and employed for other uses, such as pipeline gas (e.g., for consumer use). Water is directed into the gas synthesis module 203 along stream 213. Oxygen (e.g., in air, oxygen-enriched air, or oxygen) can also be added into the gas synthesis module.

Feed stream 206 can include C₂₊ compounds. In some cases, feed stream 206 has a C₂₊ compound concentration that is less than about 50%, 40%, 30%, 20%, 10%, 5%, or 1%.

The gas synthesis module 203 can include at least 1, 2, 3, 4, 5, 6, 7, 8, 9, 10, 20, 30, 40, or 50 gas synthesis units that are configured and adapted to generate syngas. The gas synthesis module 203 generates syngas from methane and water, and optionally oxygen, directed into the gas synthesis module 203 along streams 211 and 213, respectively. Syngas is directed from the gas synthesis module 203 to the FT module 204 along stream 214.

The FT module 204 can include at least 1, 2, 3, 4, 5, 6, 7, 8, 9, 10, 20, 30, 40, or 50 FT reactors. An FT reactor of the FT module 204 generates hydrocarbons from syngas in an FT process, as described elsewhere herein. The hydrocarbons—which can include C₂₊ compounds—and any impurities (e.g., non-C₂₊ impurities) are directed from the FT module 204 to the third separation module 205 along an FT product stream 215.

The third separation module 205 separates the hydrocarbons in stream 215 into separate components. Heavy hydrocarbons can be directed along stream 216 and light hydrocarbons can be directed along stream 217. Any methane, CO, H₂ and water can be directed along stream 218.

Heavy hydrocarbons can include hydrocarbons containing greater than or equal to 8, 10, 20, 30, 40, or 50 carbon atoms. Examples of heavy hydrocarbons include wax and diesel. Heavy hydrocarbons can be provided from the third separation module 205 in the liquid phase.

Light hydrocarbons can include hydrocarbons containing greater than or equal to about 2, 3, 4, 5, 6, 7, 8, 9, 10, or 20 carbons atoms, but fewer carbon atoms than heavy hydrocarbons. For example, light hydrocarbons can contain from two to thirty, two to fifteen, or two to ten carbon atoms. Examples of light hydrocarbons include ethane, ethylene, propane, propylene, pentane and pentenes. Light hydrocarbons can be provided from the third separation module 205 in the gas phase.

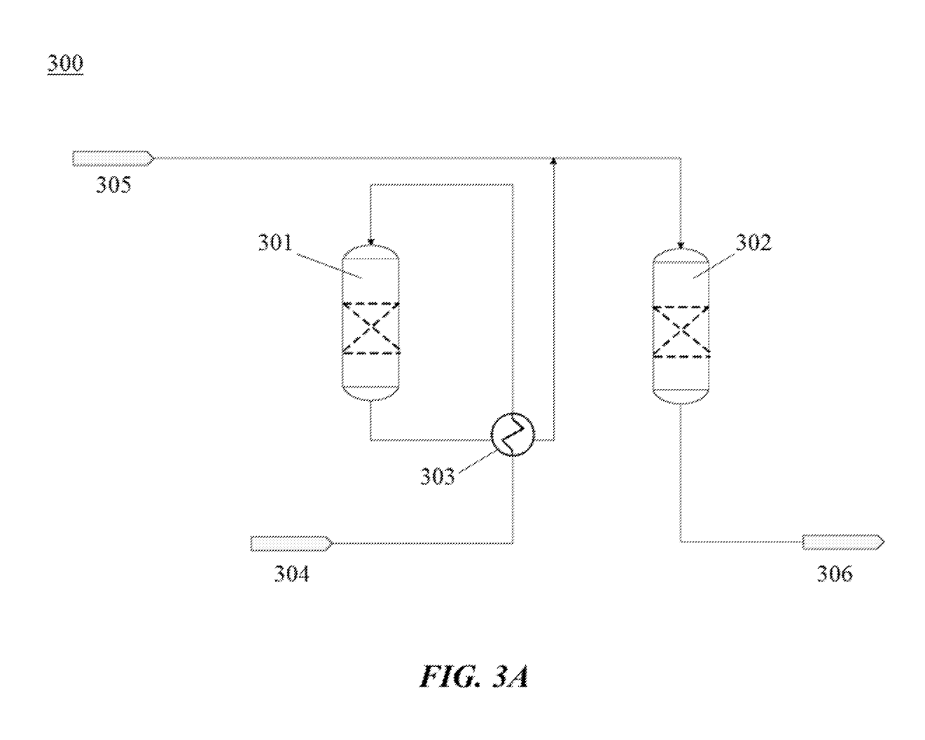

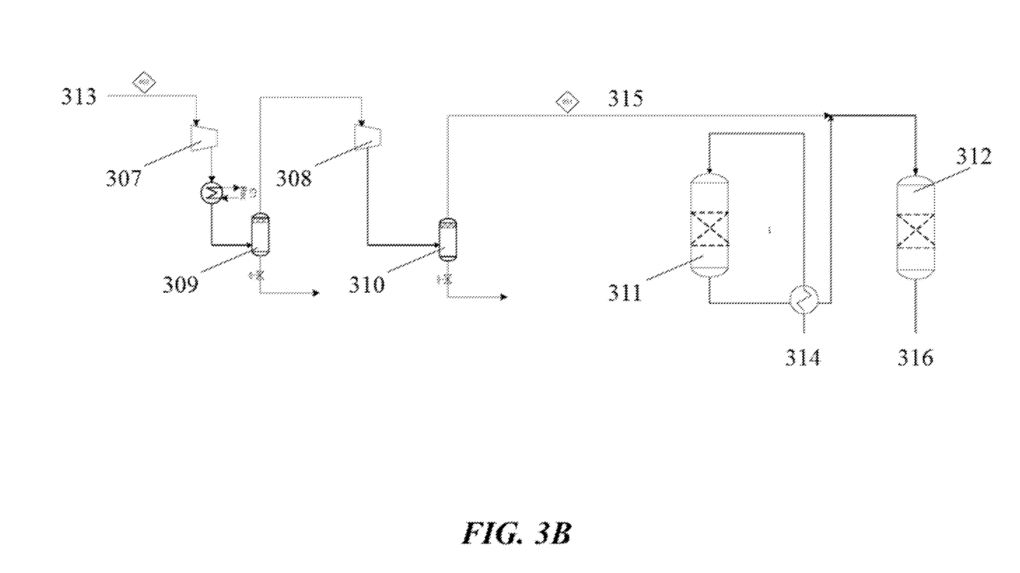

In stream 218, methane recovered from the third separation module 205 can be directed (e.g., recycled) to the gas synthesis module 203, stream 206, stream 211 and/or stream 212. CO and/or H₂ recovered from the third separation module 205 can be directed (e.g., recycled) to the FT module 204. Water recovered from the third separation module 205 can be directed (recycled) to the gas synthesis module 203. As an alternative or in addition to, any CO, CO₂ and H₂ recovered from the third separation module 205 can be directed to a methanation system (see, e.g., FIGS. 3A and 3B and the accompanying text), which can convert CO/CO₂ and H₂ to yield methane.

Stream 206 can be treated prior to being introduced to the first separation module 201. For example, stream 206 can be treated in a de-sulfurization unit 220 to remove sulfur-containing (e.g., H₂S, SO₂) chemicals 221 from the stream (e.g., before returning the sulfur depleted stream 222 to the first separation unit 201 or the gas synthesis unit 203). Such treatment can be performed with the aid of one or more separation units before the first separation module 201, such as introducing the stream 206 to a scrubbing unit (or scrubber) to remove CO₂, H₂S and/or SO₂.

Stream 216 can be subjected to additional treatment to separate one or more components of the stream. Such treatment can include introducing stream 216 to one or more separation units, such as a distillation column.

Stream 217 can be directed from the third separation module 205 to the second separation module 202 and/or to the first separation module 201. The second separation module 202 can separate hydrocarbons in stream 217 into streams each comprising a subset of the constituents of stream 217. In an example, stream 217 comprises ethane, propane and butane, and the second separation module 202 comprises a series of distillation columns that separate stream 217 into individual streams comprising ethane, propane and butane.

In some situations, the second separation module 202 can be part of a non-FT process, such as a process to generate pipeline gas from natural gas. The non-FT process can include other components that are not shown, such as heat exchangers, sensors, flow regulators (e.g., valves), and pumping systems that are configured to direct a fluid. The non-FT process can be retrofitted with the gas synthesis module 203, FT module 204 and third separation module 205. By employing the use of the second separation module 202 as well as the components (e.g., heat exchangers, sensors, flow regulators and pumping systems), a non-FT process can be configured and adapted to output a predetermined distribution of hydrocarbons, as may be tailored, for example, using the distribution of reactors in the FT module 204 and the separation that is effected using the second and third separation modules 202 and 205, respectively.

In some embodiments, the FT retrofit portion of the system (e.g., 203, 204 and 205) can share utilities with the gas-plant portion of the system (e.g., 201, 202, 220). For example, two or more of the first separation module 201, second separation module 202, and third separation module 205 can have cryogenic separation units that use a common refrigeration system, including a compressor. Heat, steam, electricity, cooling water, and/or refrigerant can be transferred between the FT retrofit portion of the system and the gas-plant portion of the system to achieve a lower overall energy use.

In some cases, the system 200 can include an oxidative coupling of methane (OCM) module. The OCM module can include one or more OCM reactor units, which can be configured and adapted to generate ethylene from methane. The OCM unit can include various stages at different temperatures, which may be suited for generating ethylene from methane. An OCM module can be separate from an FT reactor. As an alternative, an OCM unit can be integrated with an FT reactor, such as formed in the same reactor. Systems and methods for OCM are described in U.S. patent application Ser. No. 13/900,898 and U.S. patent application Ser. No. 13/936,870, which are each incorporated herein by reference in their entirety.

In some cases, the ethylene produced by the OCM module is fed to an ethylene to liquids (ETL) module. The ETL module can convert ethylene to higher molecular weight compounds (e.g., C2+ compounds) including, but not limited to gasoline, diesel fuel and aromatic chemicals. Systems and methods for ETL are described in U.S. patent application Ser. No. 14/099,614 and U.S. Patent Application No. 61/925,200, which are each incorporated herein by reference in their entirety.

In some cases, the system 200 can include a cracking module downstream of the FT module 204. The cracking module can include one or more cracking units, which can be configured and adapted to generate alkenes from alkanes, such as ethylene from ethane. The cracking unit can include various stages at different temperatures, which may be suited for generated a given olefin from an alkane. A cracking unit can be separate from an FT reactor. As an alternative, a cracking unit can be integrated with an FT reactor, such as formed in the same reactor.

FT systems and processes of the present disclosure can be suited for small scale and word scale production of hydrocarbons, such as ethylene. For example, the system 200 of FIG. 2 can be configured to generate less than or equal to about 500 kilotons per annum (KTA), 400 KTA, 300 KTA, 250 KTA, 200 KTA, 100 KTA, or 50 KTA of hydrocarbons, such as olefins (e.g. ethylene, propylene, octane), linear, cyclic, or branched alkanes (e.g. ethane, propane, butane, pentane, hexane, cyclohexane, iso-octane, etc.), aromatics (e.g. benzene, toluene, xylenes, ethylbezene, naphthenes, etc.), or blends therein such as gasoline blendstocks, distillates, natural gasoline, condensates, etc. As another scale, the system 200 can be configured to generate greater than about 50 KTA, 100 KTA, 200 KTA, 250 KTA, 300 KTA, 400 KTA, 500 KTA, or 1000 KTA of a hydrocarbon.

In some cases, the Fischer-Tropsch unit and the fractionation unit are located in close proximity to each other (e.g., within about 10 miles, 5 miles, 1 mile, 1000 feet, or 200 feet).

### Methanol to Gasoline (MTG) Systems

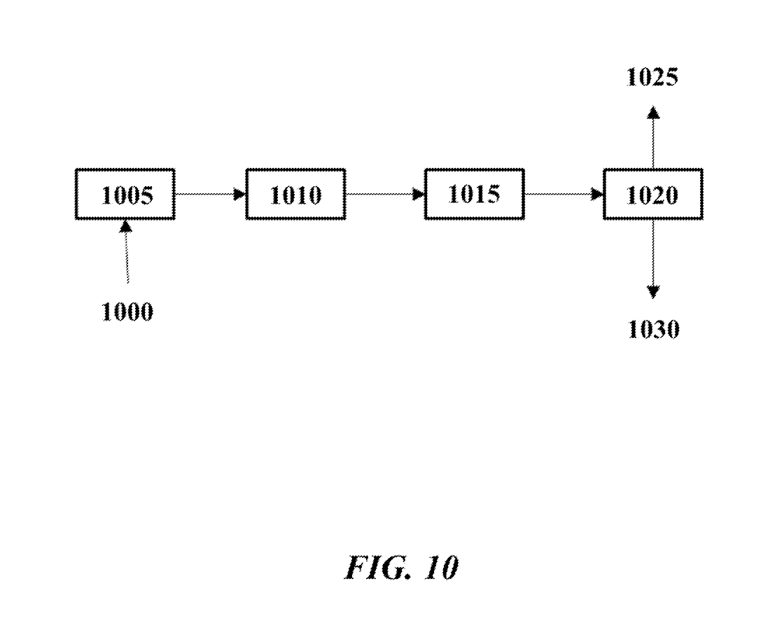

In some cases, the natural gas can be converted to gasoline via the methanol to gasoline (MTG) process. As shown in FIG. 10, methane (e.g., from natural gas) can be converted to syngas in a syngas production reactor 1005. The syngas can be converted to methanol in a methanol production reactor 1010. The methanol can be converted to gasoline or other compounds in a gasoline production module 1015. The gasoline can be fractionated from other compounds in a separation module 1020 for the separation of gasoline 1025 from other compounds 1030. A suitable catalyst for the MTG process is a doped or undoped form of zeolite, such as a ZSM-5 catalyst. Additional details of the MTG process can be found in U.S. Pat. No. 4,404,414, which is incorporated herein by reference in its entirety.

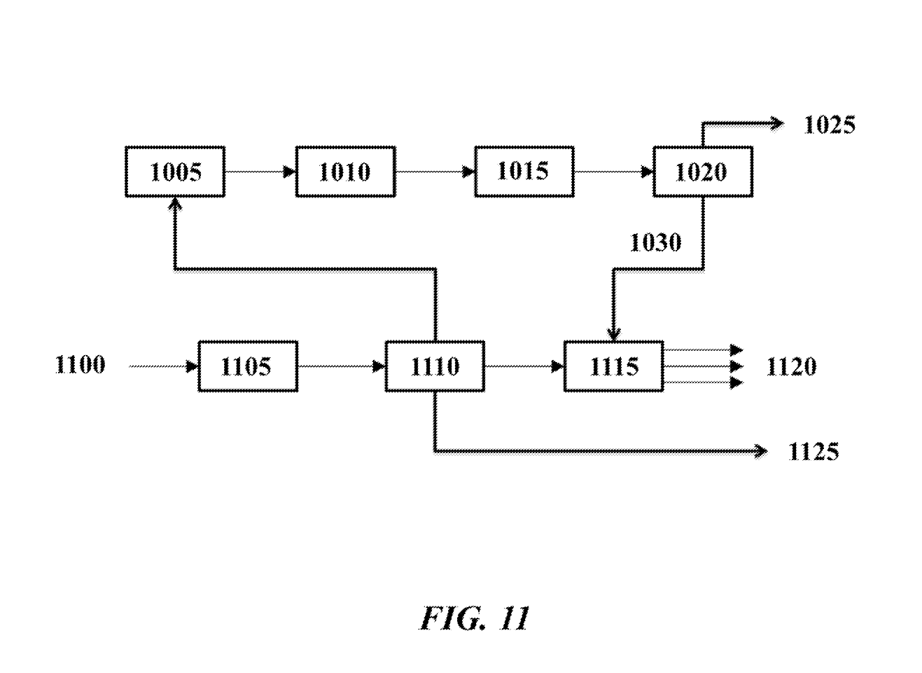

FIG. 11 shows an example of a system for converting natural gas to gasoline involving the retrofit of a natural gas processing plant with a methanol to gasoline process. In the natural gas processing plant, natural gas 1100 can be treated (e.g., to remove sulfur containing compounds, CO₂ and/or H₂O) in a gas treatment module 1105. The treated gas can be fed to a de-methanizer 1110 that separates methane from natural gas liquids (NGL) comprising C₂₊ compounds. The NGLs can be fractionated into isolated streams 1120 in an NGL product fractionation module 1115 (e.g., to produce LPG and C₅₊ products). Pipeline natural gas 1125 can be taken from the de-methanizer.

Some of the methane from the de-methanizer can be fed to the MTG process as described herein (e.g., the methane can be fed to the syngas production module 1005, followed by the methanol production module 1010, followed by the gasoline production module 1015, followed by the separation module). The separation module can separate the gasoline 1025 from other compounds 1030, which can be sent to the de-methanizer and/or the NGL product fractionation module 1115.

In some cases, the MTG process is a variant of the process called the TIGAS™ process. In conventional MTG processes, syngas is converted to methanol and some of the methanol is subsequently converted to dimethyl ether (DME) prior to the mixture of methanol and DME being converted to gasoline. In contrast, the TIGAS™ process directly converts syngas to a mixture of methanol and DME, for direct conversion to gasoline. Additional description of the TIGAS™ process can be found in U.S. Pat. No. 4,520,216, which is herein incorporated by reference in its entirety.

### Methanation Systems

Another aspect of the present disclosure provides a methanation system that can be employed to reduce the concentration of CO, CO₂ and H₂ in a given stream, such as a product stream from an FT reactor or module. The methanation system can be employed for use with any system of the present disclosure, such as the systems of FIGS. 1 and 2.

In a methanation system, CO reacts with H₂ to yield methane via CO+3H₂→CH₄+H₂O. In the methanation system, CO₂ can react with H₂ to yield methane via CO₂+4 H₂→CH₄+2 H₂O. Such processes are exothermic (ΔH=−206 kJ/mol and −178 kJ/mol, respectively) and generate heat that may be used as heat input to other process units. The methanation reaction can take place in two or more reactors in series, in some cases with intercooling. In some situations, a methanation reactor can be implemented in tandem with an FT reactor or module to increase carbon efficiency.

In some cases, to limit the heat release per unit of flow of reactants, methanation can be conducted on streams that contain CO, CO₂, H₂ and a suitable carrier gas. The carrier gas can include an inert gas, such as, e.g., N₂, He or Ar, or an alkane (e.g., methane, ethane, propane and/or butane). The carrier gas can add thermal heat capacity and significantly reduce the adiabatic temperature increase resulting from the methanation reactions.

In some examples, methane and higher carbon alkanes (e.g., ethane, propane and butane) and nitrogen are employed as carrier gases in a methanation process. These molecules can be present in an FT process, such as in an FT product stream comprising C₂₊ compounds. Downstream separation units, such as a cryogenic separation unit, can be configured to produce a stream that contains any (or none) of these compounds in combination with CO and H₂. This stream can then be directed to the methanation system.

A methanation system can include one or more methanation reactors and heat exchangers. CO, CO₂ and H₂ can be added along various streams to the one or more methanation reactors. A compressor can be used to increase the CO₂ stream pressure up to the methanation operating pressure, which can be from about 2 bar (absolute) to 60 bar, or 3 bar to 30 bar. CO₂ can be added to the inlet of the system in order to create an excess of CO₂ compared to the stoichiometric amount required to consume all the available H₂.

Given the exothermicity of the methanation reactions, a methanation system can include various methanation reactors for performing methanation. In some cases, a methanation reactor is an adiabatic reactor, such as an adiabatic fixed bed reactor. The adiabatic reactor can be in one stage or multiple stages, depending, for example, on the concentration of CO, CO₂ and H₂ in the feed stream to the methanation system. If multiple stages are used, inter-stage cooling can be performed by either heat exchangers (e.g., a stage effluent can be cooled against the feed stream or any other colder stream available in the plant, such as boiler feed water) or quenching via cold shots, i.e. the feed stream is divided into multiple streams, with one stream being directed to the first stage while each of the other feed streams being mixed with each stage effluent for cooling purposes. As an alternative, or in addition to, a methanation reactor can be an isothermal reactor. In such a case, reaction heat can be removed by the isothermal reactor by, for example, generating steam, which can enable a higher concentration of CO, CO₂ and H₂ to be used with the isothermal reactor. Apart from adiabatic and isothermal reactors, other types of reactors may be used for methanation.

FIG. 3A shows an example methanation system 300. The system 300 may be used in FT process of the present disclosure. The system 300 comprises a first reactor 301, second reactor 302 and a heat exchanger 303. The first reactor 301 and second reactor 302 can be adiabatic reactors. During use, a recycle stream 304 comprising methane, CO and H₂ (e.g., from a cryogenic separation unit) is directed to the heat exchanger 303. In an example, the recycle stream 304 comprises between about 65% and 90% (molar basis) methane, between about 5% and 15% H₂, between 1% and 5% CO, between about 0% and 0.5% ethylene, and the balance inert gases (e.g., N₂, Ar and He). The recycle stream 304 can have a temperature from about 20° C. to 30° C., and a pressure from about 2 bar to 60 bar (absolute), or 3 bar to 30 bar. The recycle stream 304 can be generated by a separation unit downstream of an FT reactor or module, such as a cryogenic separation unit.

In the heat exchanger 303, the temperature of the recycle stream 304 is increased to about 100° C. to 400° C., or 200° C. to 300° C. The heated recycle stream 304 is then directed to the first reactor 301. In the first reactor 301, CO and H₂ in the recycle stream 304 react to yield methane. This reaction can progress until all of the H₂ is depleted and/or a temperature approach to equilibrium of about 0 to 30° C., or 0 to 15° C. is achieved. The methanation reaction in the first reactor 301 can result in an adiabatic temperature increase of about 20° C. to 300° C., or 50° C. to 150° C.

Next, products from the first reactor, including methane and unreacted CO and/or H₂, can be directed along a first product stream to the heat exchanger 303, where they are cooled to a temperature of about 100° C. to 400° C., or 200° C. to 300° C. In the heat exchanger 303, heat from the first product stream 303 is removed and directed to the recycle stream 304, prior to the recycle stream 304 being directed to the first reactor 301.

Next, a portion of the heated first product stream is mixed with a CO₂ stream 305 to yield a mixed stream that is directed to the second reactor 302. The CO₂ stream 305 can be generated by a separation unit downstream of an FT reactor or module, such as a cryogenic separation unit. This can be the same separation unit that generated the recycle stream 304.

In the second reactor 302, CO and CO₂ react with H₂ to yield a second product stream 306 comprising methane. The reaction(s) in the second reactor 302 can progress until substantially all of the H₂ is depleted and/or a temperature approach to equilibrium of about 0° C. to 30° C., or 0° C. to 15° C. is achieved. The proportions of CO, CO₂ and H₂ in the mixed stream can be selected such that the second product stream 306 is substantially depleted in CO and H₂.

The first reactor 301 and the second reactor 302 can be two catalytic stages in the same reactor vessel or can be arranged as two separate vessels. The first reactor 301 and second reactor 302 can each include a catalyst, such as a catalyst comprising one or more of ruthenium, cobalt, nickel and iron. The first reactor 301 and second reactor 302 can be fluidized bed or packed bed reactors. Further, although the system 300 comprises two reactors 301 and 302, the system 300 can include any number of reactors in series and/or in parallel, such as at least 1, 2, 3, 4, 5, 6, 7, 8, 9, 10, 20, 30, 40, or 50 reactors.

Although the CO₂ stream 305 is shown to be directed to the second reactor 302 and not the first reactor 301, in an alternative configuration, at least a portion or the entire CO₂ stream 305 can be directed to the first reactor 301. The proportions of CO, CO₂ and H₂ can be selected such that the methanation product stream is substantially depleted in CO and H₂.

Methane generated in the system 300 can be employed for various uses. In an example, at least a portion of the methane can be recycled to gas synthesis module to generate syngas or employed for use as pipeline gas. As an alternative, or in addition to, at least a portion of the methane can be directed to an oxidative coupling of methane (OCM) process, which can generate higher molecular weight hydrocarbons from a feed stream comprising methane and an oxidizing agent (e.g., O₂). See, e.g., PCT/US2013/049742, which is entirely incorporated herein by reference.

FIG. 3B is a process flow diagram of an example of a methanation system that can be employed to generate ethylene. The system comprises compressors 307 and 308, separation units 309 and 310, and methanation reactors 311 and 312. The separation units 309 and 310 can be quench towers, which may separate water from a stream comprising CO and/or CO₂. During use, a stream 313 comprising CO and/or CO₂ is directed to the compressor 307 and subsequently the separator unit 309. In stream 314, CO and/or CO₂ along with H₂ are directed to the methanation reactor 311 and are reacted to form methane, which, along with any excess CO, CO₂ and H2, is subsequently directed to the methanation reactor 312, where CO and/or CO₂ provided in stream 315 is reacted with H₂ to form additional methane. The methane generated in the methanation reactors 311 and 312 is directed along stream 316. The methane in stream 316 can be, for example, recycled to an FT reactor or module.

Use of methanation systems with FT systems of the present disclosure can reduce the quantity CO and/or CO₂ that are directed to the environment, which may advantageously decrease overall greenhouse emissions from such systems. In some examples, using a methanation system, the emission of CO and/or CO₂ from an FT system can be reduced by at least about 0.01%, 0.1%, 1%, 2%, 3%, 4%, 5%, 6%, 7%, 8%, 9%, 10%, 20%, 30%, 40%, or 50%.

### Separation

The present disclosure provides various separations modules that can be employed for use with FT systems and methods of the present disclosure. The separations module can be employed to provide a desired or otherwise predetermined C₂₊ compound product distribution. Separation systems provided herein may be employed for use with various FT processes and systems of the present disclosures, such as the second separation module 202 and third separation module 205 of FIG. 2.

In natural gas processing plants or natural gas liquids (NGL) fractionation unit, methane can be separated from ethane and higher carbon-content hydrocarbons to produce a methane-rich stream that can meet the specifications of pipelines and sales gas. Such separation can be performed using cryogenic separation, such as with the aid of one or more cryogenic units, and/or by implementing one of the gas processing technologies (e.g., RSV) for maximum or optimum recovery of the NGLs.

The raw natural gas fed to gas processing plants can have a molar composition of 70% to 90% methane and 4% to 20% NGLs, the balance being inert gas(es) (e.g., CO₂ and N₂). The ratio of methane to ethane can be in the range of 5-25. Given the relatively large amount of methane present in the stream fed to cryogenic sections of gas processing plants, at least some or substantially all of the cooling duty required for the separation is provided by a variety of compression and expansion steps performed on the feed stream and the methane product stream. None or a limited portion of the cooling duty can be supplied by external refrigeration units.

There are various approaches for separating higher carbon alkanes (e.g., ethane) from natural gas, such as recycle split vapor (RSV) or any other gas processing technologies and/or gas sub-cooled process (GSP) processes, which may maximize the recovery of ethane (e.g., >99%, 98%, 97%, 96% or 95% recovery) while providing most or all of the cryogenic cooling duty via internal compression and expansion of portion of the natural gas itself (e.g., at least about 10%, 15%, 20%, 25%, 30%, 35%, 40%, or 50%). However, the application of such approach in separating alkenes (e.g., ethylene) from a stream comprising methane may result in a limited recovery in some cases when inert gas in present (e.g., provide less than 95% recovery) of the alkene product, due at least in part to i) the different vapor pressure of alkenes and alkanes, and/or ii) the presence of significant amounts of H₂ in the stream, which can change the boiling curve and, particularly, the Joule-Thomson coefficient of the methane stream that needs to be compressed and expanded to provide the cooling duty. Hydrogen can display a negative or substantially low Joule-Thomson coefficient, which can cause a temperature increase or a substantially low temperature decrease in temperature when a hydrogen-reach stream is expanded.

In some embodiments, the design of a cryogenic separation system can feature a different combination of compression/expansion steps for internal refrigeration and, in some cases, external refrigeration. The present disclosure provides a separation system comprising one or more cryogenic separation units and one or more demethanizer units. Such a system can maximize alkene recovery (e.g., provide greater than 95% recovery) from a stream comprising a mixture of alkanes, alkenes, and other gases (e.g., H₂), such as in an FT product stream.

In such separation system, the cooling duty can be supplied by a combination of expansion of the an effluent stream (e.g., feed stream to the cryogenic section) when the effluent pressure is higher than a demethanizer column; expansion of at least a portion or all of the demethanizer overhead methane-rich stream; compression and expansion of a portion of the demethanizer overhead methane-rich stream; and/or external propane, propylene or ethylene refrigeration units.

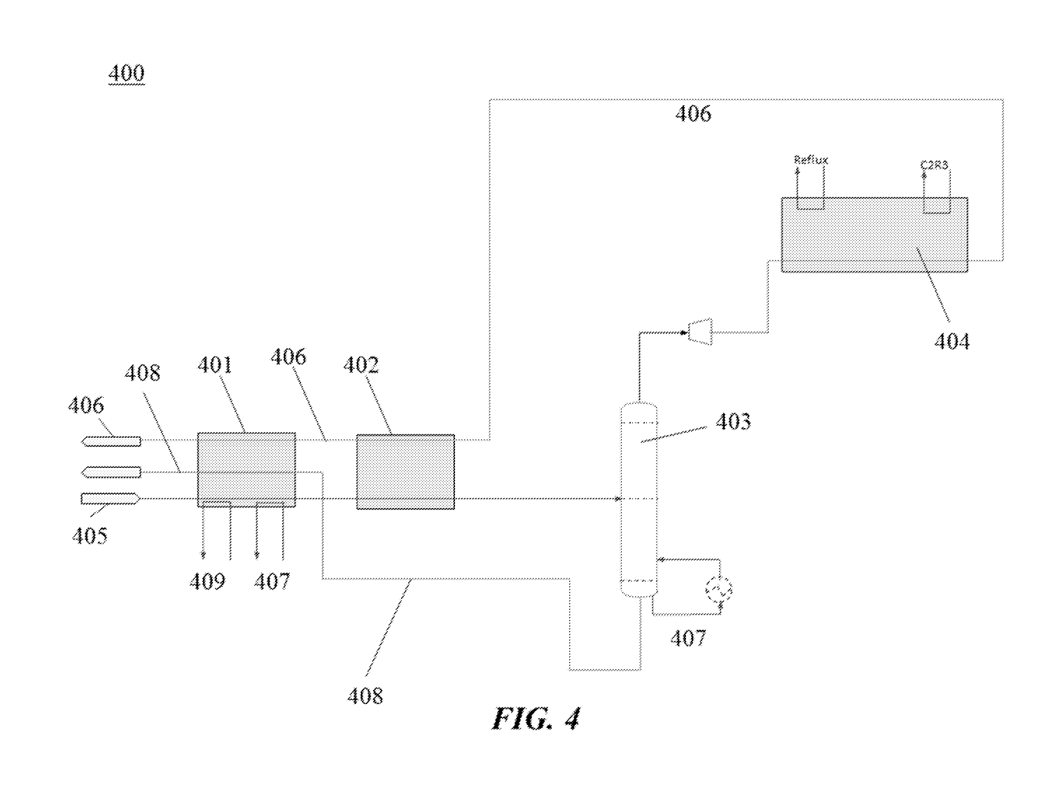

FIGS. 4-7 show various separation systems that can be employed with various systems and methods of the present disclosure, including small scale and world scale systems. FIG. 4 shows a separation system 400 comprising a first heat exchanger 401, a second heat exchanger 402, a demethanizer 403, and a third heat exchanger 404. The direction of fluid flow is shown in the figure. The demethanizer 403 can be a distillation unit or multiple distillation units (e.g., in series). In such a case, the demethanizer can include a reboiler and a condenser, each of which can be a heat exchanger. An FT product or effluent stream 405 is directed to the first heat exchanger 401 at a pressure from about 10 to 100 bar (absolute), or 20 to 40 bar. The FT effluent stream 405 can include methane and C₂₊ compounds, and may be provided in an FT product stream from an FT reactor or module (not shown). The FT effluent stream 405 is then directed from the first heat exchanger 401 to the second heat exchanger 402. In the first heat exchanger 401 and the second heat exchanger 402, the FT effluent stream 405 is cooled upon heat transfer to a demethanizer overhead stream 406, a demethanizer reboiler stream 407, a demethanizer bottom product stream 408, and a refrigeration stream 409 having a heat exchange fluid comprising propane or an equivalent cooling medium, such as, but not limited to, propylene or a mixture of propane and propylene.

The cooled FT effluent 405 can be directed to the demethanizer 403, where light components, such as CH₄, H₂ and CO, are separated from heavier components, such as ethane, ethylene, propane, propylene and any other less volatile component present in the FT effluent stream 405. The light components are directed out of the demethanizer along the overhead stream 406. The heavier components are directed out of the demethanizer along the bottom product stream 408. The demethanizer can be designed such that at least about 60%, 70%, 80%, 90%, or 95% of the ethylene in the FT effluent stream 405 is directed to the bottom product stream 408.

The demethanizer overhead stream 406 can contain at least 60%, 65%, 70%, 75%, or 80% methane. The overhead stream 406 can be expanded (e.g., in a turbo-expander or similar machine or flashed over a valve or similar device) to decrease the temperature of the overhead stream 406 prior to directing the overhead stream 406 to the second heat exchanger 402 and subsequently the first heat exchanger 401. The overhead stream 406 can be cooled in the third heat exchanger 404, which can be cooled using a reflux stream and a hydrocarbon-containing cooling fluid, such as, for example, ethylene.

The overhead stream 406, which can include methane, can be recycled, such as to a gas synthesis unit, or employed for other uses (e.g., a natural gas pipeline). In some examples, the bottom product stream, which can contain C₂₊ compounds (e.g., ethylene), can be directed to an ethylene to liquids system.

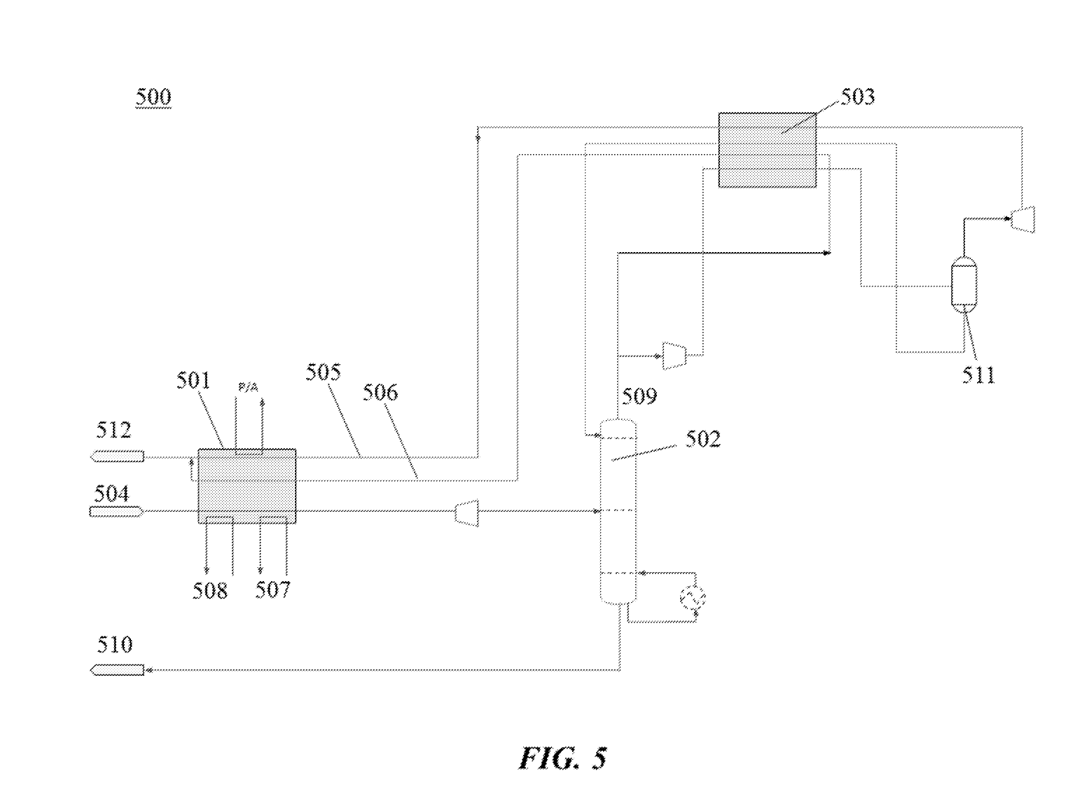

FIG. 5 shows another separation system 500 that may be employed for use with systems and methods of the present disclosure. The direction of fluid flow is shown in the figure. The system 500 comprises a first heat exchanger 501, demethanizer 502 and a second heat exchanger 503. The demethanizer 502 can be a distillation unit or multiple distillation units (e.g., in series). An FT effluent stream 504 is directed into the first heat exchanger 501. The FT effluent stream 504 can include methane and C₂₊ compounds, and may be provided in an FT product stream from an FT reactor or module (not shown). The FT effluent stream 504 can be provided at a pressure from about 10 bar (absolute) to 100 bar, or 40 bar to 70 bar. The FT effluent stream 504 can be cooled upon heat transfer to a demethanizer overhead streams 505 and 506 from the second heat exchanger 503, a demethanizer reboiler stream 507, and a refrigeration stream having a cooling fluid comprising, for example, propane or an equivalent cooling medium, such as, but not limited to, propylene or a mixture of propane and propylene. In some cases, the demethanizer overhead streams 505 and 506 are combined into an output stream 512 before or after passing through the first heat exchanger 501.

Subsequent to cooling in the first heat exchanger 501, the FT effluent stream 504 can be expanded in a turbo-expander or similar device or flashed over a valve or similar device to a pressure of at least about 5 bar, 6 bar, 7 bar, 8 bar, 9 bar, or 10 bar. The cooled FT effluent stream 504 can then be directed to the demethanizer 502, where light components (e.g., CH₄, H₂ and CO) are separated from heavier components (e.g., ethane, ethylene, propane, propylene and any other less volatile component present in the FT effluent stream 504). The light components are directed to an overhead stream 509 while the heavier components (e.g., C₂₊) are directed along a bottoms stream 510. A portion of the overhead stream 509 is directed to second heat exchanger 503 and subsequently to the first heat exchanger 501 along stream 506. A remainder of the overhead stream 509 is pressurized in a compressor and directed to the second heat exchanger 503. The remainder of the overhead stream 509 is then directed to a phase separation unit 511 (e.g., distillation unit or vapor-liquid separator). Liquids from the phase separation unit 511 are directed to the second heat exchanger 503 and subsequently returned to the demethanizer 502. Vapors from the phase separation unit 511 are expanded (e.g., in a turbo-expander or similar device) and directed to the second heat exchanger 503, and thereafter to the first heat exchanger along stream 505. The demethanizer 502 can be designed such that at least about 60%, 70%, 80%, 90%, or 95% of the ethylene in the FT effluent stream 504 is directed to the bottom product stream 510.

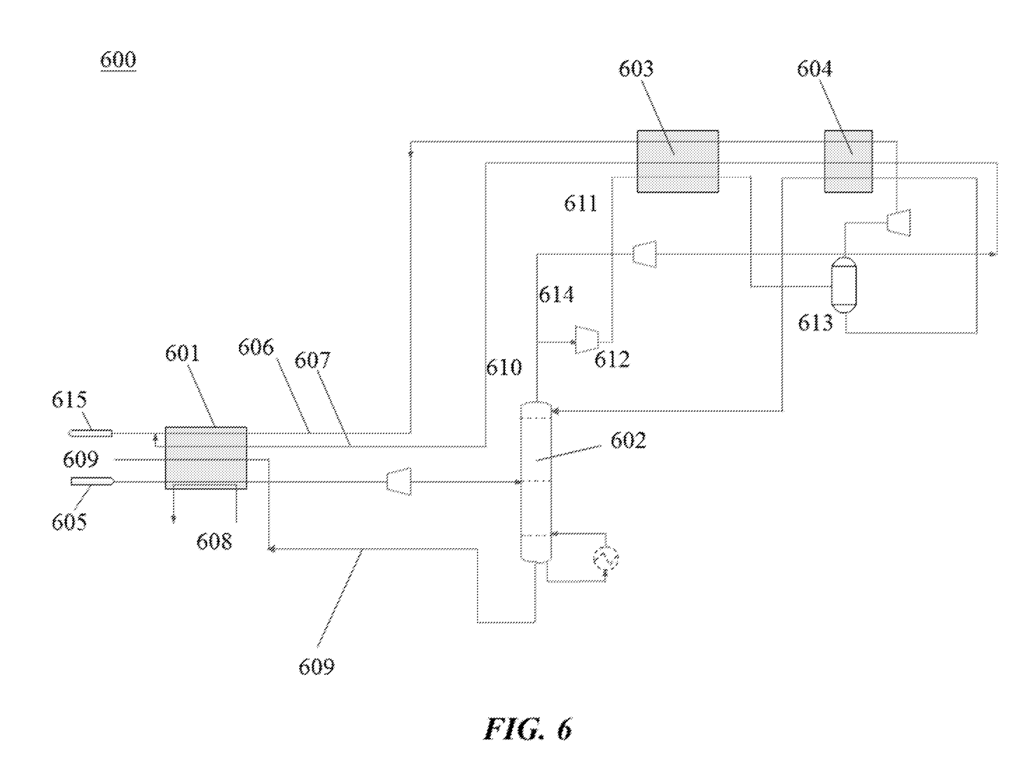

FIG. 6 shows another separation system 600 that may be employed for use with systems and methods of the present disclosure. The direction of fluid flow is shown in the figure. The system 600 comprises a first heat exchanger 601, a demethanizer 602, a second heat exchanger 603 and a third heat exchanger 604. The system 600 may not require any external refrigeration. The demethanizer 602 can be a distillation unit or multiple distillation units (e.g., in series). An FT effluent stream 605 is directed to the first heat exchanger 601 at a pressure from about 10 bar (absolute) to 100 bar, or 40 bar to 70 bar. In the first heat exchanger 601, the FT effluent stream 605 can be cooled upon heat transfer to demethanizer overhead streams 606 and 607, a demethanizer reboiler stream 608 and a demethanizer bottom product stream 609. In some cases, the demethanizer overhead streams 606 and 607 are combined into a common stream 615 before or after they are passed through the first heat exchanger 601. The FT effluent stream 605 is then expanded to a pressure of at least about 5 bar, 6 bar, 7 bar, 8 bar, 9 bar, 10 bar, or 15 bar, such as, for example, in a turbo-expander or similar machine or flashed over a valve or similar device. The cooled FT effluent stream 605 is then directed to the demethanizer 602, where light components (e.g., CH₄, H₂ and CO) are separated from heavier components (e.g., ethane, ethylene, propane, propylene and any other less volatile component present in the FT effluent stream 605). The light components are directed to an overhead stream 610 while the heavier components are directed along the bottom product stream 609. The demethanizer 602 can be designed such that at least about 60%, 70%, 80%, 90%, or 95% of the ethylene in the FT effluent stream 605 is directed to the bottom product stream 609.

The demethanizer overhead stream 610, which can contain at least 50%, 60%, or 70% methane, can be divided into two streams. A first stream 611 is compressed in compressor 612 and cooled in the second heat exchanger 603 and phase separated in a phase separation unit 613 (e.g., vapor-liquid separator or distillation column). Vapors from the phase separation unit 613 are expanded (e.g., in a turbo-expander or similar device) to provide part of the cooling duty required in heat exchangers 601, 603 and 604. Liquids from the phase separation unit 613 are sub-cooled in the third heat exchanger 604 and recycled to the demethanizer 602. A second stream 614 from the overhead stream 610 can be expanded (e.g., in a turbo-expander or similar device) to decrease its temperature and provide additional cooling to the heat exchangers 601, 603 and 604.

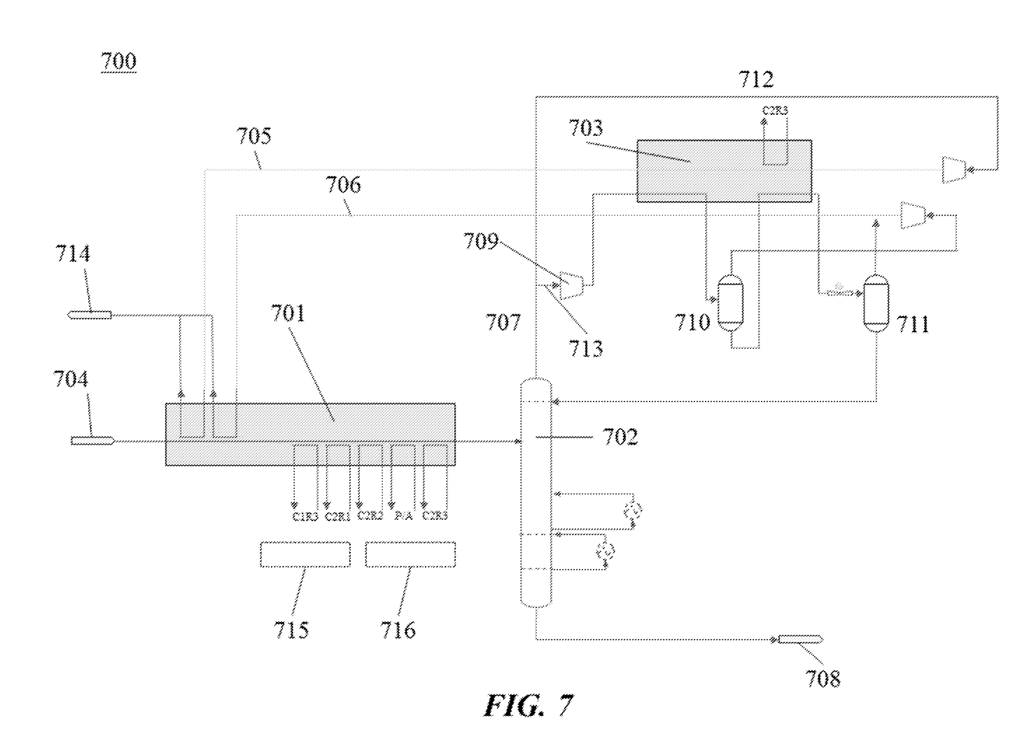

FIG. 7 shows another separation system 700 that may be employed for use with systems and methods of the present disclosure. The direction of fluid flow is shown in the figure. The system 700 comprises a first heat exchanger 701, a demethanizer 702, and a second heat exchanger 703. An FT effluent stream 704 is directed to the first heat exchanger 701 at a pressure from about 2 bar (absolute) to 100 bar, or 3 bar to 10 bar. The first heat exchanger 701 can interface with a propane refrigeration unit 715 and/or an ethylene refrigeration unit 716. In the first heat exchanger 701, the FT effluent stream 704 can be cooled upon heat transfer to demethanizer overhead streams 705 and 706, a demethanizer reboiler stream, a demethanizer pump-around stream, and various levels of external refrigeration, such as using cooling fluids comprising ethylene and propylene. In some cases, the demethanizer overhead streams 705 and 706 are combined into a single stream 714 before or after they are cooled. The cooled FT effluent stream 704 is then directed to the demethanizer 702, where light components (e.g., CH₄, H₂ and CO) are separated from heavier components (e.g., ethane, ethylene, propane, propylene and any other less volatile component present in the FT effluent stream 704). The light components are directed to an overhead stream 707 and the heavier components are directed along a bottom product stream 708. The demethanizer 702 can be designed such that at least about 60%, 70%, 80%, 90%, or 95% of the ethylene in the FT effluent stream 704 is directed to the bottom product stream 708.

The demethanizer overhead stream, which can contain at least about 50%, 60%, 70%, or 80% methane, can be divided into two streams. A first stream 713 can be compressed in a compressor 709, cooled in the second heat exchanger 703 and phase-separated in a phase separation unit 710 (e.g., distillation column or vapor-liquid separator). Vapors from the phase separation unit 710 can be expanded (e.g., in a turbo-expander or similar device) to provide part of the cooling duty required for the heat exchanger 701 and 703. Liquids from the phase separation unit 710 can be sub-cooled and flashed (e.g., over a valve or similar device), and the resulting two-phase stream is separated in an additional phase separation unit 711. Liquids from the additional phase separation unit 711 are recycled to the demethanizer 702 and vapors from the additional phase separation unit are mixed with expanded vapors from the phase separation unit 710 prior to being directed to the second heat exchanger 703.

A second stream 712 from the overhead stream 707 can be expanded (e.g., in a turbo-expander or similar device) to decrease its temperature and provide additional cooling for the heat exchanger 701 and 703. Any additional cooling that may be required for the second heat exchanger 703 can be provided by an external refrigeration system, which may employ a cooling fluid comprising ethylene or an equivalent cooling medium.

In some cases, recycle split vapor (RSV) separation can be performed in combination with de-methanization. In such a case, at least a portion of the overhead stream from a demethanizer unit (or column) may be split into at least two streams (see, e.g., FIGS. 5-7). At least one of the at least two streams may be pressurized, such as in a compressor, and directed to a heat exchanger.

In some instances, the methane undergoes a FT and/or ETL process to produce liquid fuel or aromatic compounds (e.g., higher hydrocarbon liquids) and contains molecules that have gone through methanation. In some embodiments, the compounds have been through a recycle split vapor (RSV) separation process. In some cases, alkanes (e.g., ethane, propane, butane) are cracked in a post-bed cracker.

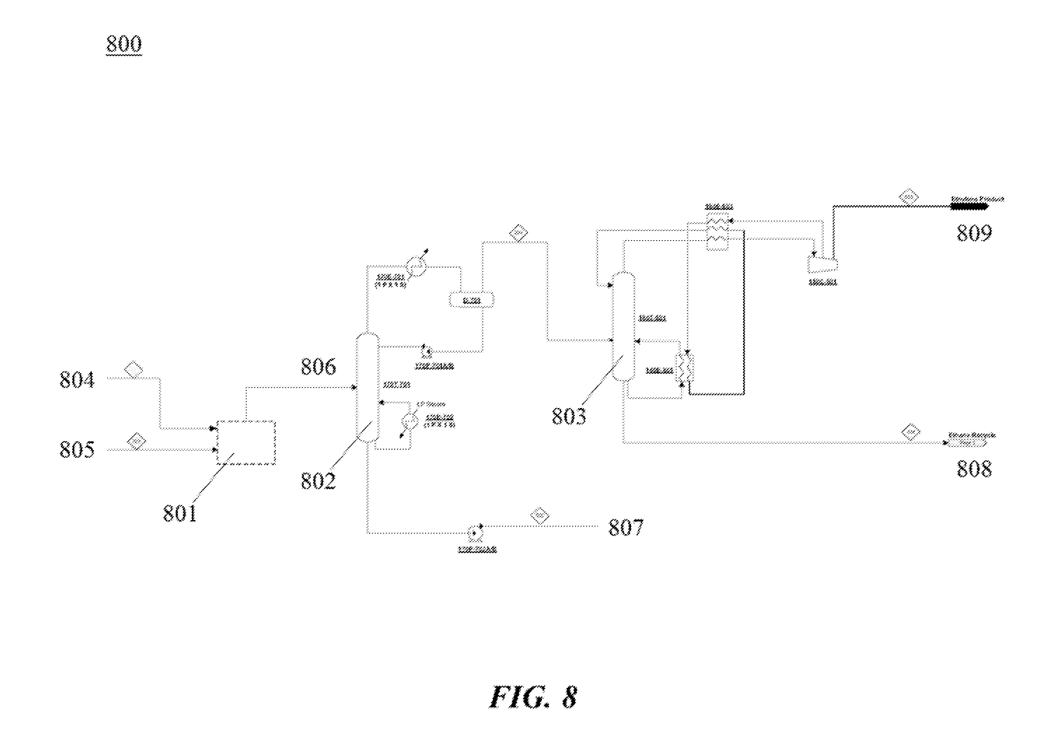

The present disclosure provides systems that can be used to tailor a product stream comprising C₂₊ compounds to include a given distribution of C₂₊ compounds. FIG. 8 is a process flow diagram of a system 800 that can be used to generate ethane and ethylene from acetylene (C₂H₂) and subsequently separate ethane from ethylene. The system 800 comprises a hydrogenation reactor unit 801, a first separation unit 802 and a second separation unit 803. The first separation unit 802 and second separation unit 803 can be distillation columns. The hydrogenation reactor unit 801 accepts a stream 804 comprising H₂ and a stream 805 comprising C₂₊ compounds, which can include acetylene, and converts any acetylene in the stream 805 to ethane and/or ethylene. The C₂₊ compounds are then directed in stream 806 to the first separation unit 802, which separates C₃₊ compounds (e.g., propane, propylene, butane, butene, etc.) from C₂ compounds (ethane and/or ethylene) in the C₂₊ compounds. The first separation unit 802 may be referred to as a demethanizer. The C₃₊ compounds are directed along stream 807 and employed for downstream use. The C₂ compounds are directed to the second separation unit 803, which separates ethane from ethylene. The second separation unit 803 may be referred to as a C₂ splitter. Ethane from the second separation unit 803 is directed along stream 808 and ethylene is directed along stream 809. Ethane can be directed to a cracking unit, which can be used to generate ethylene from ethane.

The stream 804 may be directed to a pressure swing absorption (PSA) unit (not shown) that is configured to separate H₂ from N₂. H₂ from the stream 804 may then be directed to the hydrogenation reactor 801. The stream 804 may be provided by a separation system. In situations in which a PSA is employed, the system 800 may be suitable for use in world scale olefin production. For small scale olefin production, the PSA may be precluded.

### Control Systems

The present disclosure provides computer control systems that can be employed to regulate or otherwise control methods and systems provided herein. A control system of the present disclosure can be programmed to control process parameters to, for example, effect a given product distribution, such as a higher concentration of C₂-C₁₀ hydrocarbons as compared to C₁₅₊ hydrocarbons.

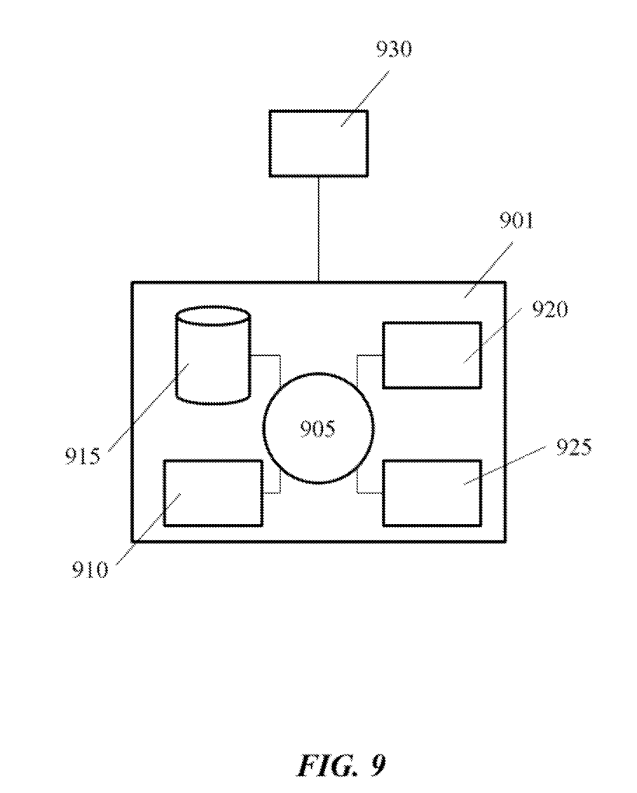

FIG. 9 shows a computer system 901 that is programmed or otherwise configured to regulate methods and systems of the present disclosure, such as regulate fluid properties (e.g., temperature, pressure and stream flow rate(s)), mixing, heat exchange and Fischer-Tropsch reactions. The computer system 901 can regulate, for example, fluid stream (“stream”) flow rates, stream temperatures, stream pressures, Fischer-Tropsch reactor temperature, Fischer-Tropsch reactor pressure, the quantity of products that are recycled, and the quantity of a first stream (e.g., methane stream) that is mixed with a second stream (e.g., air stream).

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data.

The CPU 905 can execute a sequence of machine-readable instructions, which can be embodied in a program or software. The instructions may be stored in a memory location, such as the memory 910. Examples of operations performed by the CPU 905 can include fetch, decode, execute, and writeback.

The storage unit 915 can store files, such as drivers, libraries and saved programs. The storage unit 915 can store programs generated by users and recorded sessions, as well as output(s) associated with the programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can be in communication with an FT process or system 930, including an FT reactor or module and various process elements. Such process elements can include sensors, flow regulators (e.g., valves), and pumping systems that are configured to direct a fluid.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine have a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

It will be appreciated that systems and methods described herein are provided as examples and that various alternatives may be employed. It will be further appreciated that components of systems described herein are interchangeable.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

1. A method for generating hydrocarbon compounds containing at least two carbon atoms (C₂₊ compounds), comprising:
(a) directing a natural gas feed stream comprising methane and C₂₊ compounds to at least one separation unit to separate said methane from said C₂₊ compounds, wherein said natural gas feed stream is from a non-Fischer-Tropsch process;
(b) directing at least a portion of said methane separated in (a) to a syngas unit, and in said syngas unit partially oxidizing said methane to hydrogen (H₂) and carbon monoxide (CO);
(c) directing said hydrogen and carbon monoxide to a Fischer-Tropsch unit comprising a Fischer-Tropsch catalyst, and in said Fischer-Tropsch unit reacting said hydrogen and carbon monoxide in a Fischer-Tropsch process to generate a product stream comprising C₂₊ compounds; and
(d) directing said C₂₊ compounds separated in (a) and said product stream comprising said C₂₊ compounds generated in (c) to a fractionation unit, and in said fractionation unit separating said C₂₊ compounds into individual streams.

2. The method of claim 1, wherein compounds comprising at least 10 carbon atoms are removed from said product stream prior to directing said product stream to said fractionation unit in (d).

3. The method of claim 2, wherein said compounds comprising at least 10 carbon atoms are removed using a refrigerant shared with said fractionation unit.

4. (canceled)

5. The method of claim 1, wherein said fractionation unit comprises at least one cryogenic separation unit.

6. The method of claim 5, wherein said at least one cryogenic separation unit is a propylene-based cryogenic separation unit.

7. The method of claim 1, wherein said fractionation unit comprises at least one pressure swing adsorption unit.

8.-14. (canceled)

15. The method of claim 1, further comprising directing C₂₊ compounds generated in (c) to a light hydrocarbon recovery unit, wherein said light hydrocarbon recovery unit generates tail gas.

16. The method of claim 15, further comprising directing said tail gas to said at least one separation unit.

17. The method of claim 1, further comprising directing hydrogen or carbon monoxide recovered in said fractionation unit to said Fischer-Tropsch unit.

18. The method of claim 1, further comprising, prior to (d), directing said C₂₊ compounds generated in (c) to at least one separation unit to separate said C₂₊ compounds from non-C₂₊ impurities comprising one or more of methane, H₂ and CO.

19. (canceled)

20. The method of claim 18, further comprising directing said non-C₂₊ impurities to said syngas unit or said Fischer-Tropsch unit.

21. (canceled)

22. The method of claim 1, further comprising, prior to (b), directing a portion of said methane separated from said C₂₊ compounds for use as pipeline gas.

23.-29. (canceled)

30. The method of claim 1, further comprising, prior to (a), retrofitting said non-Fischer Tropsch process with a Fischer Tropsch process comprising said syngas unit and said Fischer Tropsch unit.

31. The method of claim 30, wherein said non-Fischer-Tropsch process comprises said fractionation unit.

32. The method of claim 1, further comprising, prior to (d), separating hydrocarbon compounds containing at least 30 carbon atoms from said generated C₂₊ compounds.

33. (canceled)

34. The method of claim 1, further comprising, prior to (d), directing said product stream to a methanation unit to convert CO, CO₂ and H₂ in said product stream to methane.

35. The method of claim 1, further comprising, prior to (d), directing said product stream to a cracking unit to convert alkanes among said C₂₊ compounds to alkenes.

36.-40. (canceled)

41. A system for generating hydrocarbon compounds containing at least two carbon atoms (C₂₊ compounds), comprising:
a. at least one separation unit that (i) accepts a natural gas feed stream comprising methane and C₂₊ compounds from a non-Fischer-Tropsch process and (ii) separates said methane from said C₂₊ compounds;
b. a syngas unit downstream of and fluidically coupled to said at least one separation unit, wherein said syngas unit (i) accepts at least a portion of said methane separated in said at least one separation unit and (ii) partially oxidizes said methane to hydrogen (H₂) and carbon monoxide (CO);
c. a Fischer-Tropsch unit downstream of and fluidically coupled to said syngas unit, wherein said Fischer-Tropsch unit comprises a Fischer-Tropsch catalyst for facilitating a Fischer-Tropsch process, and wherein said Fischer-Tropsch unit (i) accepts said H₂ and said CO and (ii) reacts said H₂ and CO in said Fischer-Tropsch process to generate a product stream comprising C₂₊ compounds; and
d. a fractionation unit downstream of and fluidically coupled to said at least one separation unit and said Fischer-Tropsch unit, wherein said fractionation unit (i) accepts said C₂₊ compounds separated in said at least one separation unit and said product stream comprising said C₂₊ compounds generated in said Fischer-Tropsch unit, and (ii) separates said C₂₊ compounds into individual streams.

42. The system of claim 41, wherein said fractionation unit comprises at least one cryogenic separation unit.

43.-48. (canceled)

49. The system of claim 41, further comprising a methanation unit that converts CO, carbon dioxide (CO₂), and H₂ in said product stream to methane.

50. The system of claim 41, further comprising a cracking unit that converts alkanes among said C₂₊ compounds to alkenes.

51.-54. (canceled)
